(19) United States
(12) Patent Application Publication
(10) Pub. No.: US 2023/0104401 A1
(43) Pub. Date: Apr. 6, 2023

(54) PRESSURE MODULATED MOTOR TORQUE FOR INFUSION PUMP

(71) Applicant: Smiths Medical ASD, Inc., Plymouth, MN (US)

(72) Inventors: Henry Madden, Minnetonka, MN (US); Paul Harrison Coons, Sunfish Lake, MN (US); Marisa Furry, Prior Lake, MN (US); Kevin Krautbauer, St. Paul, MN (US)

(21) Appl. No.: 17/910,675

(22) PCT Filed: Mar. 10, 2021
(86) PCT No.: PCT/US2021/021696

§ 371 Date: Sep. 9, 2022

Related U.S. Application Data
(60) Provisional application No. 62/987,435, filed on Mar. 10, 2020.

(51) Int. Cl. A61M 5/145; A61M 5/172; A61M 5/315

(52) U.S. Cl. CPC A61M 5/1452; A61M 5/172; A61M 5/31576; A61M 2205/332

(57) ABSTRACT

An infusion pump can be configured to modulate a drive motor based on perceived infusion pressure requirements for quieter, more energy-efficient operation of the drive motor. The infusion pump can include an electrical motor having a variable output torque based on the electrical current input, a plunger head sensor configured to detect a force between a plunger driver and a plunger of a medicament container, and a control module configured to regulate the electrical current input to the electrical motor based on the detected force between the plunger driver and the plunger medicament container. The electrical current input for such an infusion pump can be incrementally reduced according to defined magnitudes of detected force.

### CROSS-REFERENCE TO RELATED APPLICATIONS

The present application claims priority to U.S. Provisional Application No. 62/987,435, filed on Mar. 10, 2020, the disclosure of which is hereby fully incorporated by reference herein.

### TECHNICAL FIELD

The present disclosure relates generally to infusion pump systems, and more particularly to systems and methods for modulating of a drive motor of an infusion pump based on a detected force.

### BACKGROUND

In the medical arts, infusion pumps have been used for managing the delivery and dispensation of a prescribed amount or dose of a drug, fluid, fluid like substance, or infusate (herein, collectively, an infusate or medicament) to patients. Infusion pumps have been used to control the volume and timing of doses among other parameters. Infusion pumps provide significant advantages over manual administration of infusates by accurately delivering infusates at rates ranging from as low as 0.01 ml/hr to as much as 1200 ml/hr, over an extended period of time. Infusion pumps are particularly useful for treating diseases and disorders that require regular pharmacological intervention, including cancer, diabetes, and vascular, neurological, and metabolic disorders. Infusion pumps also enhance the ability of healthcare providers to deliver anesthesia and manage pain.

There are many types of infusion pumps, including ambulatory, large-volume, patient controlled anesthesia (PCA), elastomeric, syringe, enteral, and insulin pumps. Depending upon the specific designs and intended uses, infusion pumps can be used to administer medication through a variety of delivery methods, including intravenously, intraperitoneally, interarterially, intradermally, subcutaneously, in close proximity to nerves, and into an inter-operative site, epidural space, or subarachnoid space. Infusion pumps are used in various settings, including hospitals, nursing homes, and other short-term and long-term medical facilities, as well as in residential care settings.

One type of infusion pump, as aforementioned, is commonly referred to as a syringe pump, in which a prefilled syringe is mechanically driven under microprocessor control to deliver a prescribed amount or dose of medicament to a patient through an infusion line or tubing in fluid connection with the prefilled syringe. Syringe pumps typically include a motor that rotates a lead screw. The lead screw in turn activates a plunger driver which forwardly pushes (or urges or otherwise acts against) a plunger within a barrel of a syringe that has been removably installed in the pump. It will be noted that the plunger driver may also travel in a reverse or opposite direction, e.g., away from a syringe. Pushing the plunger of the syringe forward thus forces the infusate outward from the syringe into the infusion line tubing and then into the patient. Examples of syringe pumps are disclosed in published PCT Application WO2016/183349, titled “High Accuracy Syringe Pumps,” and U.S. Published Patent Application No. 2017/0203032, titled “Method and Apparatus for Overload Protection in Medicament Syringe Pumps,” (assigned to Applicants of the present disclosure), both of which are hereby fully incorporated by reference herein. As used throughout this disclosure, the term “syringe pump” is intended to generally pertain to any device which acts on a syringe to controllably force infusate outwardly therefrom.

Although such syringe pumps have proven to work quite well, there is a desire to continually improve syringe pump systems. In particular, it is desirable to provide syringe pumps that are quieter in operation and consume less power than some known pumps. Although previous attempts to produce quieter, more energy-efficient infusion pumps have been made, the conventional wisdom has generally led to either mechanical isolation of the motor with the goal of producing a quieter operating pump, or the use of a lower power motor to produce a quieter, more energy-efficient infusion pump. U.S. Published Patent Application No. 2013/0123749, titled “Drug Delivery Pump Drive Using Linear Piezoelectric Motor,” (assigned to Roche Diabetes Care Inc.) discloses one such example of a quieter, more efficient infusion pump, which uses a lower power linear piezoelectric motor as the drive element.

While these examples of infusion pumps do generally provide quieter operation and lower energy consumption, particularly as compared to pumps having traditional electric motor-based drives, such pumps can be prone to stall or otherwise become interrupted in their operation in high infusion pressure conditions (e.g., 14-18 psi). Because pressure increases are not uncommon in various pump operating environments and situations, there is a need to provide a more consistent performance with a decreased potential for interruption of treatment during infusion. Thus, while it may be desirable to produce a quieter, more energy-efficient infusion pump, the pump must be large or powerful enough to meet or exceed reliability and dependability standards by providing steady state operations during a range of infusion pressures without deleterious stalling or interruption of operation. The present disclosure addresses these concerns.

### SUMMARY OF THE DISCLOSURE

Embodiments of the present disclosure provide an apparatus and method of controlling the power input to a drive motor capable of handling a full range of infusion pressures according to a sensed infusion pressure, thereby operating the drive motor in a quieter, more energy efficient manner when the infusion pressures are low enough to afford such operation. For example, in an embodiment, the apparatus and method can employ an electric motor having an electrical input of between about 0.1 and about 1.0 amperes (A), with an ability to reduce electrical input based on a detected force between a syringe pump plunger driver and a plunger of a medicament container or syringe. In another embodiment, an electrical motor having an electrical input of between about 0.175 A and about 0.7 A may be implemented. Accordingly, in some embodiments, the input current can be significantly reduced during steady-state normal operating conditions (e.g., an infusion pressure of less than about 8 psi), thereby providing quieter operation and lower energy consumption in comparison to traditional electric drive systems. Improved energy efficiency may be especially desirable when operating an infusion pump on battery power.

An embodiment of the present disclosure provides an infusion pump configured to modulate an electrical current input of a drive motor based on perceived infusion pressure requirements. The infusion pump includes an electrical motor, a force sensor, and a control module. The electrical motor can have a variable output torque based on an electrical current input. The force sensor is configured to detect a force between a plunger driver of the pump and the plunger within a medicament container. The control module is configured to adjust the electrical current input to the electrical motor based on force detected by the force sensor.

In an embodiment, electrical current input for an electric motor in an infusion pump can be incrementally reduced according to defined magnitudes of force detected by a plunger driver sensor. In such an embodiment, the electrical current input can be maintained: at the maximum rated power input of the electric motor when the detected force is greater than or equal to about 80 N, at about 75% of the electric motor's maximum rated power input when the detected force is less than about 80 N, at about 50% of the electric motor's maximum rated power input when the detected force is less than about 65 N, and at about 25% of the electric motor's maximum rated power when the detected force is negligible. In one embodiment the electrical current input for the electric motor can be reduced according to a nonlinear function of the detected force along a continuous curve.

Another embodiment of the present disclosure provides a method of operating an infusion pump including detecting a force between a plunger driver of the infusion pump and a plunger within a medicament container, and modulating an electrical current to an electrical drive motor based on the detected force between the plunger driver and the plunger in the medicament container.

In another embodiment, the present disclosure provides an infusion pump configured to modulate an electrical current input of a drive motor based at least part on a linear rate of travel of a plunger driver of the infusion pump. The infusion pump includes an electrical motor, a plunger head sensor, and a control module. The electrical motor can have a variable output torque based on an electrical current input. The plunger head sensor is configured to detect the linear rate of travel of the plunger driver during operation as the plunger driver pushes on a medicament container such as a plunger of a syringe. The control module is configured to adjust the electrical current input to the electrical motor based on the linear rate of travel detected by the plunger head sensor.

In another embodiment, the present disclosure provides an infusion pump configured to modulate an electrical current input of a drive motor based on both a force between a plunger driver and a medicament container as detected by a force sensor, and a linear rate of travel of a plunger driver of the infusion pump.

In an embodiment, the present disclosure provides an infusion pump configured to modulate an electrical current input of a drive motor based on perceived infusion pressure requirements. The infusion pump comprises a pump housing defining a syringe receptacle shaped and sized to accept loading of a syringe, an electrical motor having a variable output torque based on the electrical current input, and a syringe drive assembly. The syringe drive assembly includes a lead screw operably coupled with the electrical motor, a plunger driver operably coupled with the lead screw and linearly movable in response to rotation of the electrical motor, the plunger driver configured to push against a plunger of the syringe, and a force sensor configured to detect a force between the plunger driver and the plunger of the syringe. The infusion pump further comprises a control module configured to regulate the electrical current input to the electrical motor based on the detected force between the plunger driver and the plunger of the syringe.

In an embodiment, the present disclosure provides a method of operating an infusion pump. The method comprises providing, by a control module, an electrical current input to an electrical motor of the infusion pump so as to cause a plunger driver of the infusion pump to push against a plunger of a syringe installed in the infusion pump, wherein the electrical motor includes a variable output torque based on the electrical current input. The method further comprises detecting a force between the plunger driver and the plunger of the syringe using a force sensor, and modulating, by the control module, the electrical current input to the electrical motor based on the detected force between the plunger driver and the plunger of the syringe.

In an embodiment, the present disclosure provides an infusion pump configured to modulate an electrical current input of a drive motor based on perceived infusion pressure requirements. the infusion pump comprises a pump housing defining a syringe receptacle shaped and sized to accept loading of a syringe, an electrical motor having a variable output torque based on the electrical current input, and a syringe drive assembly. The syringe drive assembly includes a lead screw operably coupled with the electrical motor, a plunger driver operably coupled with the lead screw and linearly movable in response to rotation of the electrical motor, the plunger driver configured to push against a plunger of the syringe, a force sensor configured to detect a force between the plunger driver and the plunger of the syringe, and a plunger head sensor configured to detect a linear rate of travel of the plunger driver. The infusion pump further comprises a control module configured to regulate the electrical current input to the electrical motor based on the detected force between the plunger driver and the plunger of the syringe, the control module further configured to regulate the electrical current input to the electrical motor based on the linear rate of travel of the plunger driver as detected by the plunger head sensor, wherein the electrical current input is incrementally reduced according to defined magnitudes of detected force, and wherein a maximum rated power input for the drive motor is less than about 1.0 A.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a front perspective view depicting a syringe pump, according to an embodiment.

FIG. 2 is a perspective view of a syringe plunger driver assembly, according to an embodiment.

FIG. 3 is a block diagram depicting components of a syringe pump, according to an embodiment.

FIG. 4 is a graph depicting a motor stall curve, input modulation curve, and safety factor, according to an embodiment.

FIG. 5 is a graph depicting noise criteria rating against flow rate for two different electrical current inputs, according to an embodiment.

FIG. 6 is a flow chart depicting a method of operating an infusion pump, according to an embodiment.

FIG. 7 is a flow chart depicting a method of operating an infusion pump, according to an embodiment.

FIG. 8 is a flowchart depicting a method of operating an infusion pump, according to an embodiment.

FIG. 8A is another flowchart depicting a method of operating an infusion pump, according to an embodiment.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

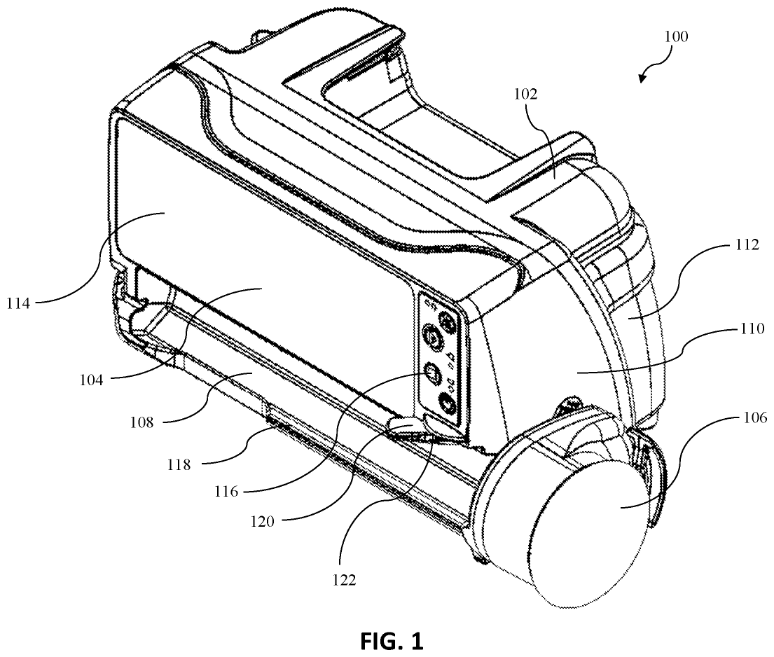

Referring to FIG. 1, a syringe pump 100 is depicted in accordance with an embodiment of the disclosure. The syringe pump 100 can include a housing 102, a user interface 104, a drive assembly (syringe plunger driver assembly) 106, and a medicament container receptacle 108. In some embodiments, the housing 102 can include a front housing assembly 110 and a rear housing assembly 112, configured to generally form a protective shell surrounding internal components of the syringe pump 100.

The user interface 104 can include a display screen 114 and a keypad 116. The display screen 114 can be any suitable graphical user interface (GUI) display for use in controlling the syringe pump 100. For example, in an embodiment, the display screen 114 can be a multicolor liquid crystal display (LCD), dot matrix display, organic light-emitting diode (OLED) display and/or any other device capable of visually delivering and/or accepting information. In some embodiments, the display screen 114 can be appropriately sized to enable a display of drug and/or patient information, infusate delivery parameters, and other information. In an embodiment, the display screen 114 can measure approximately 180 mm×73 mm; although other display screen sizes are also contemplated. In some embodiments, the display screen 114 can be configured to display instructional video, for example, to aid caregivers in proper maintenance and use of the syringe pump 100. In some embodiments, the display screen can include touchscreen capabilities, thereby enabling certain commands and/or instructions to be received by the display screen 114.

The keypad 116 can be located adjacent to the display screen 114 and can present a variety of buttons and indicator lights. In some embodiments, pushbuttons requiring physical mechanical actuation can be used on the keypad 116 to receive certain user commands, including on-off power; audible alarm mute; and starting and stopping the delivery of infusate. Additional or fewer buttons on the keypad 116 are also contemplated. Physical mechanical actuation buttons, for primary and redundant purposes, provide increased safety and reliability to operators in cases where touchscreen capabilities of a display screen 114 are not properly functioning, or are otherwise difficult to correctly manipulate. Accordingly, the inclusion of a user interface 104 having both a display screen 114 and a keypad 116 provide the flexibility and usefulness of a screen interface, as well as the enhanced safety and reliability of physical control buttons.

The medicament container receptacle 108 can be defined between a portion of the front housing assembly 110 and a syringe ledge 118. The medicament container receptacle 108 can be configured as an elongate cavity extending across the front of the syringe pump 100 configured to accept medicament containers (e.g., syringes) of a variety of shapes and sizes when loaded into the syringe pump 100. In some embodiments, the medicament container receptacle 108 can provide a cavity in the syringe pump 100 that remains open to the front of the syringe pump 100, such that a loaded medicament container is readily and sustainably visible.

In some embodiments, the medicament container receptacle 108 is located below the display screen 114 of the user interface 104. Location of the medicament container receptacle 108 below the user interface 104 can be advantageous, as any unintended fluid leakage from the syringe may naturally flow downwards due to gravity and away from the user interface 104, thereby avoiding potential damage to electronic and/or mechanical features of the user interface 104. Accordingly, the medicament container receptacle 108 can be somewhat spatially isolated, advantageously, from the remainder of the syringe pump 100 in the event of damage to the medicament container or other leakage during loading, unloading or manipulation. Additionally, because the display screen 114 is located above the medicament container receptacle 108, the display screen 114 is generally not visibly obstructed by the presence of a medicament container loaded in the medicament container receptacle 108. That is, the location of the display screen 114 above the medicament container receptacle 108 enables unobscured visibility of both the medicament container and the display during operation of the syringe pump 100.

In some embodiments, the syringe pump 100 can further include a barrel clamp device 120, located within the medicament container receptacle 108 and/or generally underneath the user interface 104. The barrel clamp device 120 can be configured to shift and rotate relative to the front housing assembly 110, for example, along an axis generally orthogonal to an axis of the medicament container receptacle 108, thereby enabling capture of a barrel of a medicament container between the barrel clamp device 120 and a portion of the syringe ledge 118. In some embodiments, the barrel clamp device 120 can include a barrel clamp sensor 122 (as depicted in FIG. 1) configured to electronically sense when the barrel of a medicament container is captured between the barrel clamp device 120 and a portion of the syringe ledge 118, and therefore when a medicament container is loaded into the medicament container receptacle 108. In some embodiments, the barrel clamp sensor 122 can include a linear potentiometer configured to sense the degree to which the barrel clamp device 120 is extended or displaced from the front housing assembly 110, and therefore the approximate diameter of a medicament container loaded into the medicament container receptacle 108. In some embodiments, the sensed approximate diameter of the barrel can be used for medicament container (or syringe) characterization.

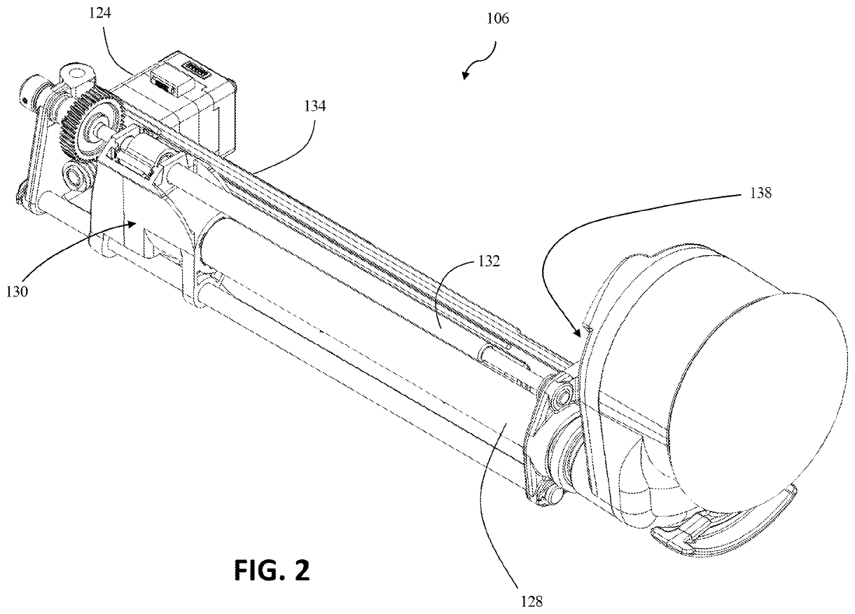

Referring to FIG. 2, the syringe plunger driver assembly 106 can include a motor 124, drivetrain assembly 126 and a plunger driver 128. In an embodiment, the motor 124 can be a stepper motor and encoder configured to rotate in discrete step increments when electrical command pulses are applied. In some embodiments, the motor 124 can be configured to detect motor stalls and rotational slowing below nominal motor rotational speeds.

The motor 124 can be operably coupled to the drivetrain assembly 126, which can be configured to convert the rotational output of the motor 124 to a linear movement (or actuation) for use by the plunger driver 128. For example, in an embodiment, the drivetrain assembly 126 can include a carriage assembly 130, lead screw 132, and drivetrain chassis 134. In operation, rotation of the lead screw 132 (e.g., via the motor 124) can force the carriage assembly 130 to shift, translate or otherwise move relative to the drivetrain chassis 134. In some embodiments, the drivetrain assembly 126 can further include a plunger head sensor 136 (e.g., a linear potentiometer) (as depicted in FIG. 2) configured to determine positional data of the carriage assembly 130 relative to the drivetrain chassis 134. The plunger driver 128 can be operably coupled to the carriage assembly 130 and can include a force sensor 138 configured to sense a force magnitude acting upon a thumb press (or plunger) of a syringe loaded into the syringe pump 100. In some embodiments, the force sensor 138, plunger head sensor 136 and barrel clamp sensor 122 can gather and utilize data individually or cooperatively for improved operational characterization.

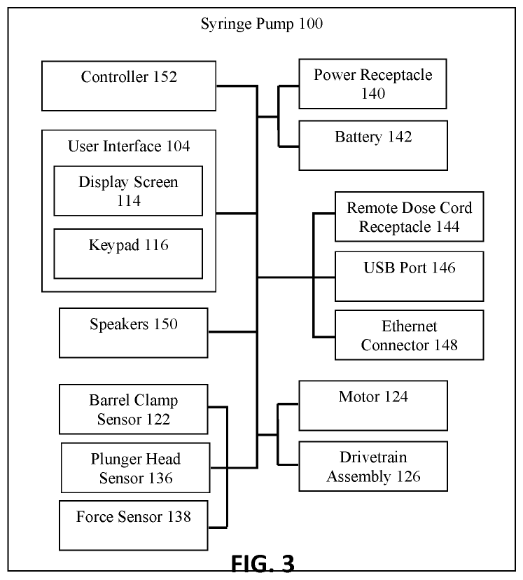

Referring to FIG. 3, a block diagram of the syringe pump 100 is depicted in accordance with an embodiment of the disclosure. As previously described, the syringe pump 100 can include a user interface 104 that can include a display screen 114 and keypad 116. The syringe pump 100 can further include a power receptacle 140, battery 142, remote dose cord receptacle 144, USB port 146, ethernet connection 148, one or more speakers 150, controller 152, motor 124, and drivetrain assembly 126.

The controller 152 can be configured to control operation of the motor 124 and drivetrain assembly 126. The controller 152, which can be powered by the power receptacle 140 and/or the battery 142, can include one or more processors and/or a memory. In some embodiments, the controller 152 is in electrical communication with the user interface 104, the remote dose cord receptacle 144, USB port 146 and/or the ethernet connection 148, for the purpose of receiving information from and transmitting information to users of the syringe pump 100. In an embodiment, the controller 152 can be in electrical communication with the barrel clamp sensor 122, plunger head sensor 136, and force sensor 138, and can be configured to receive data sensed by sensors 122, 136 and 138 for further processing.

In one embodiment, data received from sensors 122, 136, and/or 138 can be used by the controller 152 to modulate a torque output of the motor 124. Modulation of the torque output of the motor 124 can provide more control over the noise output and energy efficiency of syringe pump 100. A low torque output of the motor 124 can be relatively energy efficient and quiet while a high torque output promotes infusion rate consistency and inhibits stalling across a range of infusion pressures. For example, in an embodiment, the motor 124 output can be modulated (e.g., via the controller 152) based on a force sensed between the plunger driver 128 and the plunger of a syringe, as measured by the force sensor 138. In another embodiment, the motor 124 output can be modulated (e.g., via the controller 152) based on a linear rate of travel of plunger driver 128, as measured by plunger head sensor 136.

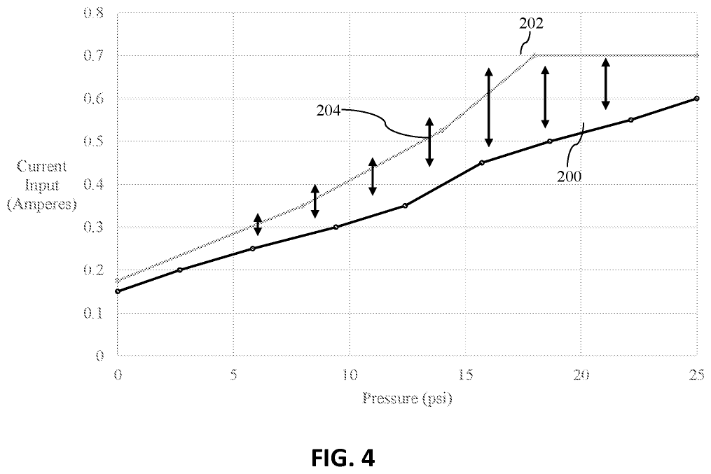

FIG. 4 depicts a motor stall curve 200, graphically representing the stall threshold (e.g., the point at which a motor 124 of a given size stalls) across a range of electrical power inputs and corresponding system pressures. As depicted, the y-axis represents the electrical current input to motor 124 in amperes, while the x-axis represents the pressure in pounds per square inch as measured by force sensor 138, for example. Below the motor stall curve 200, the motor 124 will stall (e.g., stop rotating) because the torque required by the system pressure is greater than the maximum torque generated by the motor 124 at the corresponding electrical input, resulting in an inconsistent infusion rate (e.g., where the motor slows below its nominal speed or intermittently stalls) and/or interruption of the infusion (e.g., where the motor stalls for a prolonged period of time).

FIG. 4 further depicts an electrical current input modulation curve 202, according to an embodiment of the disclosure. Accordingly, in an embodiment, the current input can be modulated between a minimum of about 0.175 A and a maximum of about 0.7 A; although other magnitudes of current input are also contemplated, depending upon the size and requirements of the motor 124. The y-axis gap between the motor stall curve 200 and the electrical current input modulation curve 202 can represent a safety factor 204, which in an embodiment can generally be configured to increase in magnitude between about 0 psi and about 18 psi.

With reference to Table 1 below, the electrical current input can be incrementally increased and/or decreased according to defined force magnitude thresholds (or defined ranges of force magnitude), as detected by the force sensor 138. For example, when a negligible amount of force is detected by sensor the force sensor 138, the controller 152 can regulate the current input of the motor 124 to about 25% of its maximum rated power input (e.g., about 0.175 A). When the force detected by the force sensor 138 is within a first threshold range (e.g., between a force greater than about 0 N and about 64.1 N), the controller 152 can regulate the current input of the motor 124 to about 50% of its maximum rated power input (e.g., about 0.35 A). When the force detected by the force sensor 138 is within a second threshold range (e.g., between a force greater than about 64.1 N and about 82.5 N) the controller 152 can regulate the current input of the motor 124 to about 75% of its maximum rated power (e.g., about 0.525 A). When the force detected by the force sensor 138 is above a third threshold (e.g., a force greater than about 82.5 N) the controller 152 can regulate the current input of the motor to 100% of its maximum rated power (e.g., about 0.7 A). The use of specific current inputs, motor output percentages and applied forces on force sensor 138 are for exemplary purposes only and should not be considered limiting; other current inputs, motor output percentages and applied forces on force sensor 138 are also contemplated.

TABLE 1

| Software | System | Applied | Motor | Motor |
|---|---|---|---|---|
| Logic | Pressure | Force on | Current | Current |
| Points: | (psi): | Sensor (N): | (Amps): | (%) |
| 1.1 | 18 | >82.5 | 0.7 | 100% |
| 0.1 | 14 | 64.1-82.5 | 0.525 | 75% |
| 1.0 | 8 | 0-64.1 | 0.35 | 50% |
| 0.0 | N/A | N/A | 0.175 | 25% |

It is to be appreciated and understood that Table 1 is an example listing of incrementally modulated electrical current input. Accordingly, in some embodiments such as that depicted by Table 1, the electrical current input is incrementally modulated in steps (e.g., corresponding to prospective motor outputs of about 25%, 50%, 75%, and 100%) based on estimated infusion pressures (e.g., corresponding to about 0 psi, 8 psi, 14 psi, and 18 psi), which can either be sensed directly via a fluid pressure sensor contacting the infusate (not depicted) or via the force sensor 138. In other embodiments, the electrical current input can be modulated according to a (linear or nonlinear) function of the detected fluid pressure and/or force (e.g., via force sensor 138) between the plunger driver 128 and a plunger of a syringe in the syringe pump 100.

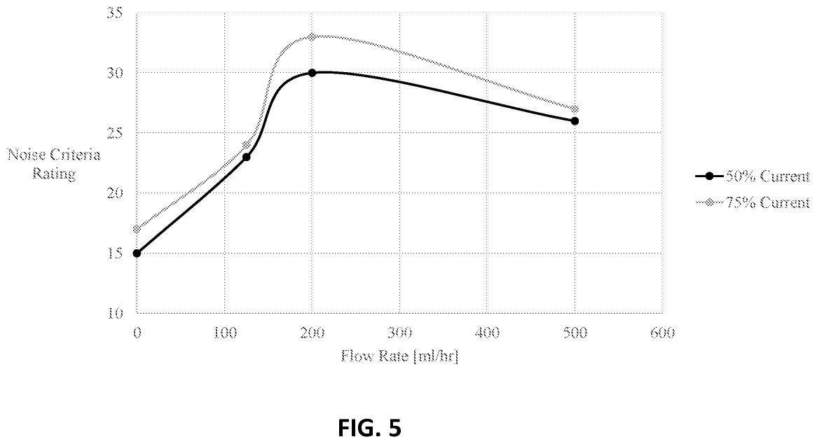

In addition to improved power efficiency, reducing the electrical current input to the motor 124 has the effect of reducing the overall noise produced by the motor 124 during an infusion and/or treatment protocol across a range of flow rates. FIG. 5 depicts a noise criteria rating curve, graphically representing measured noise criteria ratings across a range of infusion flow rate outputs. As depicted, the y-axis represents the noise criteria (NC) rating, while the x-axis represents the flow rate of infusate in milliliters per hour. Accordingly, as depicted, reducing the input current from about 75% to about 50% has the effect of variably reducing the noise criteria rating across the spectrum of infusion flow rate outputs.

In some embodiments, the controller 152 can alternately or additionally use inputs from the barrel clamp sensor 122 and/or the plunger head sensor 136 in modulation of the electrical current input to the motor 124. In an embodiment, the safety factor 204 can be increased and/or decreased based on a perceived syringe size (as determined by, for example, the barrel clamp sensor 122). For example, if it is determined that the infusion is to be administered via a relatively large syringe (e.g., a syringe of greater than about 20 mL), the safety factor 204 can be multiplied by a constant, or a constant can be added to the safety factor 204, effectively increasing the safety factor 204 in the anticipation of larger and potentially more rapid fluctuations in system pressures. Conversely, if it is determined that the infusion is to be administered via a relatively small syringe (e.g., a syringe of less than about 10 mL), the safety factor 204 can be divided by a constant, or a constant can be subtracted from the safety factor, effectively decreasing the safety factor 204 for quieter performance and improved electrical efficiency. Conversely, increasing the safety factor for a syringe below a certain size and decreasing the safety factor for a syringe above a certain size is also contemplated.

In an embodiment, the safety factor can be increased and/or decreased based on a perceived travel of the plunger within the syringe (as determined by the plunger head sensor 136). For example, if it is determined that the syringe is filled to its maximum capacity (or otherwise that the syringe pump 100 is in an early stage of an infusion treatment), the safety factor 204 can be multiplied by a constant, or a constant can be added to the safety factor 204, effectively increasing the safety factor 204. If, on the other hand, it is determined that the infusion treatment has been ongoing for some determined quantity of time, and no motor stalls have occurred, the safety factor 204 can be divided by a constant, or a constant can be subtracted from the safety factor, effectively decreasing the safety factor 204 for quieter performance and improved electrical efficiency. It is also contemplated that the safety factor 204 can be incrementally or continuously increased as the infusion treatment progresses.

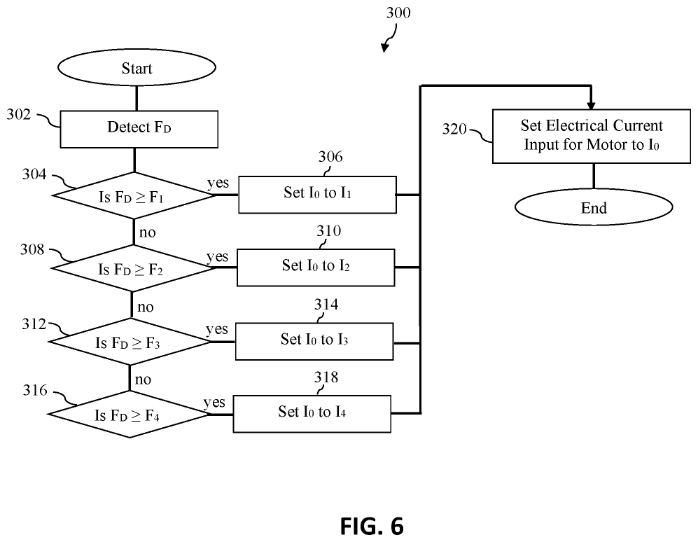

Referring to the example of Table 1 above and to FIG. 6, a flowchart depicting a method 300 of operating an infusion pump in a quieter, more energy-efficient manner is depicted in FIG. 6 in accordance with an embodiment of the disclosure. At 302, a force (F_{D}) between the plunger driver 128 and the plunger of a medicament container can be measured (e.g., via force sensor 138). Thereafter, F_{D} can be received by and stored within the memory of controller 152 for further processing. At 304, F_{D} can be compared to a first defined force value (F₁) (e.g., about 80 N). If F_{D} is greater or equal to F₁, then at 306, an ideal current input (I₀) can be set to a first defined current input value (I₁) (e.g., about 0.7 A). Alternatively, if F_{D} is less than F₁, then the method 300 can proceed to 308.

At 308, F_{D} can be compared to a second defined force value (F₂) (e.g., about 65 N). If F_{D} is greater or equal to F₂, then at 310, I₀ can be set to a second defined current input value (I₂) (e.g., about 0.5 A). Alternatively, if F_{D} is less than F₂, then the method 300 can proceed to 312. At 312, F_{D} can be compared to a third defined force value (F₃) (e.g., about 0.1 N). If F_{D} is greater or equal to F₃, then at 314, I₀ can be set to a third defined current input value (I₃) (e.g., about 0.3 A). Alternatively, if F_{D} is less than F₃, then the method 300 can proceed to 316. At 316, F_{D} can be compared to a fourth defined force value (F₄) (e.g., a negligible force). If F_{D} is greater than or equal to F₄, then at 318, I₀ can be set to a fourth defined current input value (I₄) (e.g., about 0.2 A). Id can be stored within the memory of controller 152. Once the ideal current input I₀ has been set to a defined current input value then the method 300 can proceed to 320, where the electrical current input to the motor 124 can be set to substantially meet the ideal current input I₀.

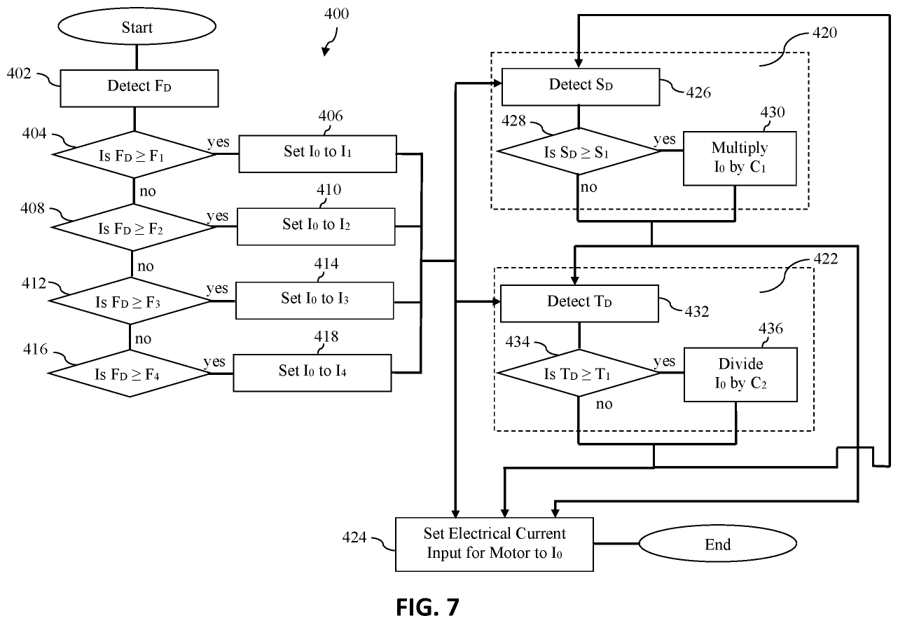

Referring to FIG. 7, a flowchart depicting a method 400 of operating an infusion pump in a quieter, more energy-efficient manner is depicted in accordance with an embodiment of the disclosure. At 402, a force (F_{D}) between the plunger driver 128 and the plunger of a medicament container can be measured (e.g., via force sensor 138). Thereafter, F_{D} can be received by and stored within the memory of controller 152 for further processing. At 404, F_{D} can be compared to a first defined force value (F₁) (e.g., about 80 N). If F_{D} is greater or equal to F₁, then at 406, an ideal current input (I₀) can be set to a first defined current input value (I₁) (e.g., about 0.7 A). Alternatively, if F_{D} is less than F₁, then the method 400 can proceed to 408.

At 408, F_{D} can be compared to a second defined force value (F₂) (e.g., about 65 N). If F_{D} is greater or equal to F₂, then at 410, I₀ can be set to a second defined current input value (I₂) (e.g., about 0.5 A). Alternatively, if F_{D} is less than F₂, then the method 400 can proceed to 412. At 412, F_{D} can be compared to a third defined force value (F₃) (e.g., about 0.1 N). If F_{D} is greater or equal to F₃, then at 414, I₀ can be set to a third defined current input value (I₃) (e.g., about 0.3 A). Alternatively, if F_{D} is less than F₃, then the method 400 can proceed to 416. At 416, F_{D} can be compared to a fourth defined force value (F₄) (e.g., a negligible force). If F_{D} is greater than or equal to F₄, then at 418, I₀ can be set to a fourth defined current input value (I₄) (e.g., about 0.2 A). I₀ can be stored within the memory of controller 152. Method 400 can optionally include block operations 420 and 422. If block operations 420 and 422 are not included, then the method 400 can proceed to 424, where the electrical current input to the motor 124 can be set to substantially meet the ideal current input I₀.

In an embodiment, if block operation 420 is included in method 400, the ideal current input can be further adjusted based on medicament container size. According to block operation 420, at 426, a medicament container size (S_{D}) (e.g., diameter) can be determined (e.g., via barrel clamp sensor 122). Thereafter, S_{D} can be received by and stored within the memory of controller 152 for further processing. At 428, S_{D} can be compared to a defined size value (S₁). If S_{D} is greater or equal to S₁, then at 430, I₀ can be multiplied by a first constant (C₁). Method 400 can then proceed to 424, where the electrical current input to the motor 124 can be set to substantially meet the ideal current input I₀. Alternatively, if the block operation 422 is included in the method 400 and has yet to be considered, the method 400 can proceed to optional block operation 422.

In an embodiment, if block operation 422 is included in method 400, the ideal current input can be further adjusted based on medicament container plunger travel distance. According to block operation 422, at 432, a medicament container plunger travel distance (T_{D}) can be determined (e.g., via plunger head sensor 136). Thereafter, T_{D} can be received by and stored within the memory of controller 152 for further processing. At 434, T_{D} can be compared to a defined travel distance (T₁). If T_{D} is greater or equal to T₁, then at 436, I₀ can be divided by a second constant (C₂). Method 400 can then proceed to 424, where the electrical current input to the motor 124 can be set to substantially meet the ideal current input I₀. Alternatively, if the block operation 420 is included in the method 400 and has yet to be considered, the method 400 can proceed to optional block operation 420.

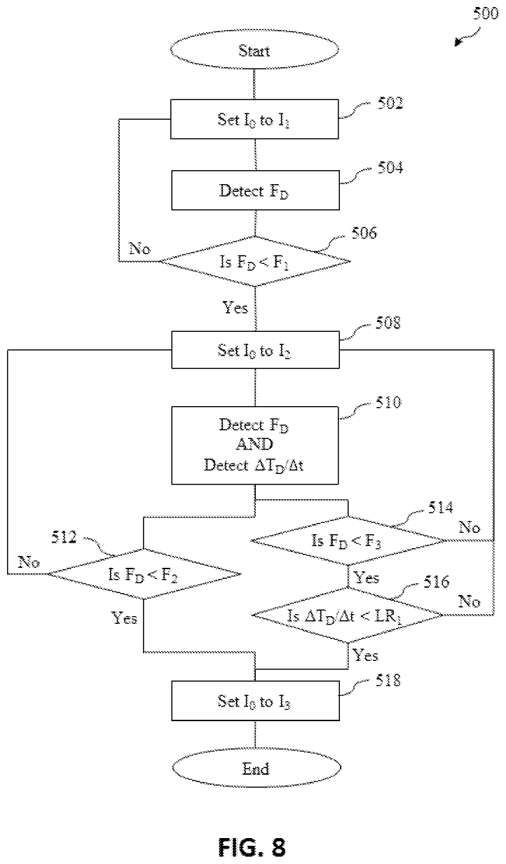

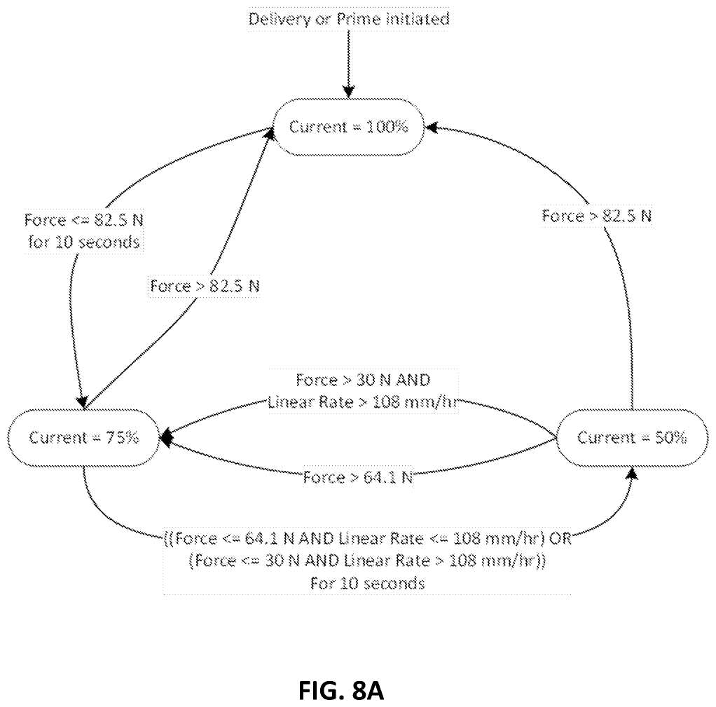

Referring to FIG. 8, a flowchart depicting a method 500 of operating an infusion pump in a quieter, more energy-efficient manner is depicted in accordance with an embodiment of the disclosure. At 502, an ideal current input (I₀) can be set to a first defined current input value (I₁) (e.g., about 0.7 A or about 100% of its maximum rated power input).

At 504, a force (F_{D}) between the plunger driver 128 and the plunger of a medicament container can be measured (e.g., via force sensor 138). Thereafter, F_{D} can be received by and stored within the memory of controller 152 for further processing. At 506, F_{D} can be compared to a first defined force value (F₁) (e.g., about 82.5 N). If F_{D} is less than F₁, then at 508, the ideal current input (I₀) can be set to a second defined current input value (I₂) (e.g., about 0.525 A or about 75% of its maximum rated power input). Alternatively, if F_{D} is greater than or equal to F₁, then the method 500 can revert to 502.

At 510, a force (F_{D}) between the plunger driver 128 and the plunger of a medicament container can be measured (e.g., via force sensor 138), and a linear rate (e.g., ΔT_{D}/Δt) can be measured (e.g., via plunger head sensor 136). Thereafter, F_{D} and ΔT_{D}/Δt can be received by and stored within the memory of controller 152 for further processing. At 512, F_{D} can be compared to a second defined force value (F₂) (e.g., about 64.1 N). If F_{D} is less than F₂, then at 518, the ideal current input (I₀) can be set to a third defined current input value (I₃) (e.g., about 0.35 A or about 50% of its maximum rated power input). Alternatively, if F_{D} is greater than or equal to F₂, then the method 500 can revert to 508.

Additionally, at 514 F_{D} can be compared to a third defined force value (F₃) (e.g., about 30 N). If F_{D} is less than F₃, then the method 500 can proceed to 516. Alternatively, if F_{D} is greater than or equal to F₃, then the method 500 can revert to 508. At 516, ΔT_{D}/Δt can be compared to a first linear rate value (LR₁) (e.g., about 108 mm/hr). If ΔT_{D}/Δt is less than LR₁, then at 518, the ideal current input (I₀) can be set to the third defined current input value (I₃) (e.g., about 0.35 A or about 50% of its maximum rated power input). Alternatively, if ΔT_{D}/Δt is greater than or equal to LR₁, then the method 500 can revert to 508. Accordingly, in some embodiments, the infusion pump can utilize a linear rate of travel of the plunger as a proxy for a motor rotational rate, for example where available motor torque decreases with an increase in motor rate.

FIG. 8A depicts a specific example of the method 500 depicted in FIG. 8.

It should be understood that the individual steps used in the methods of the present disclosure may be performed in any order and/or simultaneously, as long as the disclosure remains operable. Furthermore, it should be understood that the apparatus and methods of the present disclosure can include any number, or all, of the described embodiments, as long as the disclosure remains operable.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An infusion pump configured to modulate an electrical current input of a drive motor, the infusion pump comprising:
a pump housing defining a syringe receptacle shaped and sized to accept loading of a syringe;
an electrical motor having a variable output torque based on the electrical current input;
a syringe drive assembly, including:
a lead screw operably coupled with the electrical motor;
a plunger driver operably coupled with the lead screw and linearly movable in response to rotation of the electrical motor, the plunger driver configured to push against a plunger of the syringe; and
a force sensor configured to detect a force between the plunger driver and the plunger of the syringe; and
a control module configured to regulate the electrical current input to the electrical motor based on the detected force between the plunger driver and the plunger of the syringe.

2. The infusion pump of claim 1, wherein the electrical current input is incrementally reduced according to defined magnitudes of detected force.

3. The infusion pump of claim 1, wherein the electrical current input is maintained at a maximum rated power input when the detected force is greater than or equal to about 80 N.

4. The infusion pump of claim 3, wherein the maximum rated power input is about 0.7 A.

5. The infusion pump of claim 1, wherein the electrical current input is reduced to about 75% of its maximum rated power input when the detected force is less than about 80 N.

6. The infusion pump of claim 1, wherein the electrical current input is reduced to about 50% of its maximum rated power input when the detected force is less than about 65 N.

7. The infusion pump of claim 1, wherein the electrical current input is reduced to about 25% of its maximum rated power input when the detected force is negligible.

8. The infusion pump of claim 1, wherein the electrical current input is reduced according to a nonlinear function of the detected force along a continuous curve.

9. The infusion pump of claim 1, the syringe drive assembly further comprising:
a plunger head sensor configured to detect a linear rate of travel of the plunger driver,
wherein the control module is further configured to regulate the electrical current input to the electrical motor based on the linear rate of travel of the plunger driver as detected by the plunger head sensor.

10. A method of operating an infusion pump, the method comprising:
providing, by a control module, an electrical current input to an electrical motor of the infusion pump so as to cause a plunger driver of the infusion pump to push against a plunger of a syringe installed in the infusion pump, wherein the electrical motor includes a variable output torque based on the electrical current input;
detecting a force between the plunger driver and the plunger of the syringe using a force sensor; and
modulating, by the control module, the electrical current input to the electrical motor based on the detected force between the plunger driver and the plunger of the syringe.

11. The method of claim 10, further comprising:
detecting a linear rate of travel of the plunger driver using a plunger head sensor; and
modulating, by the control module, the electrical current input to the electrical motor based on the detected linear rate of travel of the plunger driver.

12. The method of claim 10, wherein the electrical current input is modulated at a maximum rated power input when the detected force is greater than or equal to about 80 N.

13. The method of claim 12, wherein the maximum rated power input is about 0.7 A.

14. The method of claim 10, wherein the electrical current input is modulated at about 75% of its maximum rated power input when the detected force is less than about 80 N.

15. The method of claim 10, wherein the electrical current input is modulated at about 50% of its maximum rated power input when the detected force is less than about 65 N.

16. The method of claim 10, wherein the electrical current input is modulated at about 25% of its maximum rated power input when the detected force is negligible.

17. The method of claim 11, wherein the electrical current input is modulated at about 75% of its maximum rated power input when the detected force is greater than about 30 N and the detected rate of travel is greater than about 108 millimeters per hour.

18. The method of claim 11, wherein the electrical current input is modulated at about 50% of its maximum rated power input when the detected force is less than about 64 N and the detected rate of travel is less than about 108 millimeters per hour.

19. An infusion pump configured to modulate an electrical current input of a drive motor, the infusion pump comprising:
a pump housing defining a syringe receptacle shaped and sized to accept loading of a syringe;
an electrical motor having a variable output torque based on the electrical current input;
a syringe drive assembly, including:
a lead screw operably coupled with the electrical motor;
a plunger driver operably coupled with the lead screw and linearly movable in response to rotation of the electrical motor, the plunger driver configured to push against a plunger of the syringe;
a force sensor configured to detect a force between the plunger driver and the plunger of the syringe; and
a plunger head sensor configured to detect a linear rate of travel of the plunger driver; and
a control module configured to regulate the electrical current input to the electrical motor based on the detected force between the plunger driver and the plunger of the syringe, the control module further configured to regulate the electrical current input to the electrical motor based on the linear rate of travel of the plunger driver as detected by the plunger head sensor,
wherein the electrical current input is incrementally reduced according to defined magnitudes of detected force, and
wherein a maximum rated power input for the drive motor is less than about 1.0 A.
